(19) United States
(12) Patent Application Publication
(10) Pub. No.: US 2016/0354038 A1
(43) Pub. Date: Dec. 8, 2016

(54) REMOVING MOTION-RELATED ARTIFACTS IN HEART RATE MEASUREMENT SYSTEMS USING ITERATIVE MASK ESTIMATION IN FREQUENCY-DOMAIN

(71) Applicant: ANALOG DEVICES, INC., NORWOOD, MA (US)

(72) Inventors: SEFA DEMIRTAS, MALDEN, MA (US); ROBERT ADAMS, ACTON, MA (US); JEFFREY G. BERNSTEIN, MIDDLETON, MA (US); NOAH DANIEL STEIN, SOMERVILLE, MA (US); DAVID WINGATE, PROVO, UT (US)

(73) Assignee: ANALOG DEVICES, INC., NORWOOD, MA (US)

(21) Appl. No.: 15/170,373

(22) Filed: Jun. 1, 2016

Related U.S. Application Data
(60) Provisional application No. 62/170,386, filed on Jun. 3, 2015.

(51) Int. Cl. A61B 5/00; A61B 5/0245; A61B 5/0255; A61B 5/024

(52) U.S. Cl. CPC A61B 5/7214; A61B 5/02416; A61B 5/02438; A61B 5/721; A61B 5/725; A61B 5/0245; A61B 5/0255; A61B 2562/0204; A61B 2562/0223; A61B 2562/029; A61B 2562/0247

(57) ABSTRACT

Heart rate monitors are plagued by noisy photoplethysmography (PPG) data, which makes it difficult for the monitors to output a consistently accurate heart rate reading. Noise is often caused by motion. Using known methods for processing accelerometer readings that measure movement to filter out some of this noise may help, but not always. The present disclosure describes an improved filtering approach, referred to herein as an iterative frequency-domain mask estimation technique, based on using frequency-domain representation (e.g. STFT) of PPG data and accelerometer data for each accelerometer channel to generate filters for filtering the PPG signal from motion-related artifacts prior to tracking frequency of the heartbeat (heart rate). Implementing this technique leads to more accurate heart rate measurements.

### CROSS-REFERENCE TO RELATED APPLICATIONS

This application claims the benefit of and priority from U.S. Provisional Patent Application Ser. No. 62/170,386 filed 3 Jun. 2015 entitled “REMOVING MOTION-RELATED ARTIFACTS USING ITERATIVE FREQUENCY-DOMAIN HEART RATE MEASUREMENT TECHNIQUE,” which is incorporated herein by reference in its entirety.

### TECHNICAL FIELD OF THE DISCLOSURE

The present invention relates to the field of digital signal processing, in particular to digital signal processing for reducing motion-related artifacts in a signal used for tracking a heartbeat frequency in a noisy environment.

### BACKGROUND

Modern electronics are ubiquitous in healthcare. For example, monitoring devices often include electronic components and algorithms to sense, measure, and monitor living beings. Monitoring equipment can measure vital signs such as respiration rate, oxygen level in the blood, heart rate, and so on. Not only are monitoring devices used in the clinical setting, monitoring devices are also used often in sports equipment and consumer electronics.

One important measurement performed by many of the monitoring equipment is heart rate, typically measured in beats per minute (BPM). Athletes use heart rate monitors to get immediate feedback on a workout, while health care professionals use heart rate monitors to monitor the health of a patient. Many solutions for measuring heart rate are available on the market today. For instance, electronic heart rate monitors can be found in the form of chest straps and watches. However, these electronic heart rate monitors are often not very accurate, due to a high amount of noise present in the signals provided by the sensors of these monitors. The noise is often caused by the fact that the user is moving and also by the lack of secure contact between the monitor and the user. This noisy environment often leads to an irregular, inaccurate or even missing readout of the heart rate.

### BRIEF DESCRIPTION OF THE DRAWING

To provide a more complete understanding of the present disclosure and features and advantages thereof, reference is made to the following description, taken in conjunction with the accompanying figures, wherein like reference numerals represent like parts, in which:

FIG. 1 shows an illustrative heart rate monitoring apparatus and a portion of a living being adjacent to the heart rate monitor, according to some embodiments of the disclosure.

FIG. 2 illustrate a system view of a heart rate monitoring apparatus, according to some embodiments of the disclosure;

FIG. 3 illustrates an exemplary flow diagram of a method for tracking a heartbeat frequency present in one or more input signals provided by one or more sensors in a noisy environment, according to some embodiments of the disclosure;

FIG. 4 illustrates an exemplary flow diagram of a method for filtering input signals prior to tracking a heartbeat frequency present in the input signals, according to some embodiments of the disclosure;

FIG. 5 illustrates an exemplary flow diagram of using a PPG signal and three accelerometer channels to obtain a HR mask and applying the HR mask to obtain a filtered signal, according to some embodiments of the disclosure;

FIG. 6 illustrates contribution from individual sources to each time-frequency bin of a PPG channel, according to some embodiments of the disclosure;

FIG. 7 illustrates an example of iterative mask estimation mechanism, according to some embodiments of the disclosure;

FIG. 8 illustrates a schematic of a source model, according to some embodiments of the disclosure;

FIG. 9 illustrates an NMF source model, according to some embodiments of the disclosure;

FIG. 10 illustrates an NMF source model during training and separation, according to some embodiments of the disclosure;

FIG. 11 illustrates a constant source model, according to some embodiments of the disclosure;

FIG. 12 illustrates an identity source model, according to some embodiments of the disclosure; and

FIG. 13 illustrates an example of iterative mask estimation mechanism using color information, according to some embodiments of the disclosure.

### DESCRIPTION OF EXAMPLE EMBODIMENTS OF THE DISCLOSURE

Overview

Heart rate monitors are plagued by noisy photoplethysmography (PPG) data, which makes it difficult for the monitors to output a consistently accurate heart rate reading. Noise is often caused by motion. Using known methods for processing accelerometer readings that measure movement to filter out some of this noise may help, but not always. The present disclosure describes improved filtering approaches, referred to herein as iterative mask estimation techniques, based on using frequency-domain representation (e.g. STFT) of PPG data and accelerometer data for each accelerometer channel to generate filters for filtering the PPG signal from motion-related artifacts prior to tracking frequency of the heartbeat (heart rate). Implementing these techniques leads to more accurate heart rate measurements.

Understanding Issues of Noisy Environment of Heart Rate Monitors

Heart rate monitors are often in direct contact with the skin of a living being. The monitors passively track or measure heart rate by sensing one or more aspects of the skin adjacent to the heart rate monitor. Due to the passive nature of such measurements, the sensor data can be affected by many sources of noise which severely affects the ability of the heart rate monitor to determine an accurate heartbeat. These sources of noise can include external interference to the sensor, internal noise of the sensor and/or heart rate monitor, motion causing disruptions in the sensor's capability in measuring the aspects of the skin, etc. Furthermore, heart rate monitors are affected by variability in the skin of different living beings and the variability of the skin and environment during the use of the heart rate monitor. All these different sources and issues have adverse impact on the heart rate monitor's ability to extract an accurate heart rate.

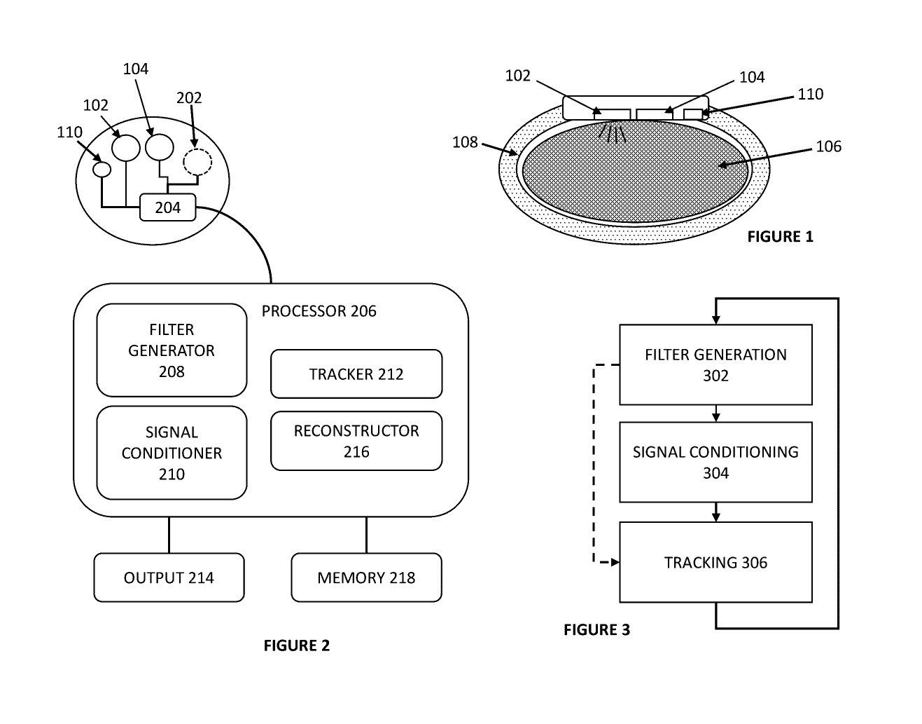

FIG. 1 shows an illustrative heart rate monitoring apparatus and a portion of a living being adjacent to the heart rate monitor, according to some embodiments of the disclosure. In particular, the FIGURE shows a cross section to illustrate the monitoring apparatus's spatial relationship with the portion of the living being. In this exemplary heart rate monitoring setup, a method of photoplethysmography (PPG) is used, where the heart rate is measured passively or indirectly based on changes in light absorption in the skin as blood is pushed through the arteries. Changes in blood volume as blood is pumped through the arteries results in a variation in the amount of received light, which is translated into electrical pulses by an optical sensor. The pulses in the signal can then be used in extracting a heart rate.

Heart rate monitoring apparatus described herein are not limited to the particular example shown in FIG. 1. Although the disclosure does not describe other types of heart rate monitors in detail, one skilled in the art would appreciate that these challenges are also applicable in other types of heart rate monitors or other types of devices providing heart rate monitoring functions, or even devices utilizing other types of sensing mechanism. Furthermore, the continued process of measuring, following, extracting, determining, or sensing the heart rate (or some other varying frequency) over time is referred to as “tracking a varying frequency”, within the context of the disclosure.

Specifically, FIG. 1 illustrates an exemplary heart rate monitoring apparatus having a light source 102 and an optical sensor 104. The light source can emit light within a range of wavelengths suitable for the application. In some embodiments, the light source 102 and the optical sensor 104 can be provided separately, or a light source 102 can be biased to function as an optical sensor 104. For instance, a red LED can be used as a red light source and a red optical detector. In some embodiments, both the light source 102 and optical sensor 104 can be provided nearby each other in a housing or member of the heart rate monitoring apparatus or in any suitable configuration where the optical sensor 104 can measure absorption of light (as generated by the light source 102) by the part 106 of the living being. The light source shines a light onto a part 106 of a living being 106 and the optical sensor 104 measures light incident onto the optical sensor 104, which can include light being reflected from the part 106 as well as ambient light. Various parts of the living being can be used as part 106, e.g., a finger, an arm, a forehead, an ear, chest, a leg, a toe, etc., as long as changes in the volume of blood can be measured relatively easily. The part 106 can in some cases be internal to the body of the living being.

Generally speaking, if the heart rate monitoring apparatus can be affixed to the part 106 of the living being securely and maintain relatively stable contact with the part 106 during use, the input signal provided by the optical sensor would exhibit very little noise and the heart rate can be easily extracted. However, in many scenarios, the heart rate monitoring apparatus is not securely affixed to the part 106 (even with the use of part 108 involving a band, a strap, adhesive, or other suitable attachments), or having the apparatus securely adhered or attached to the part 106 is not desirable or comfortable for the living being. Even when sensor is securely connected, motion can affect signal quality greatly because of blood rushing in and out of the veins during motion by large amounts, and because of tendons, tissue and bones moving around under the skin itself and changing amount of reflected light. In these scenarios, the signal provided by the optical sensor 104 is usually affected by noise from ambient light, artifacts caused by motion of the heart rate monitoring apparatus, or by some other noise source. As a result, correctly detecting the heart rate in these non-ideal scenarios, i.e., in a noisy environment, can be challenging. Attempting to detect the heart rate based on a noisy signal can result in irregular or erroneous heart rate readings.

To address this issue, some heart rate monitoring apparatuses include a mechanism which discards certain portions of data if the data is deemed unusable for tracking the heart rate. The mechanism can include an accelerometer 110 to measure the motion of the apparatus to assess whether the input signal is likely to be too degraded by motion artifacts to be relied upon for heart rate determination. In those cases, the accelerometer reading can cause the apparatus to discard data or freeze the heart-rate readout when the accelerometer 110 senses too much motion. Another approach may be to use the accelerometer data to estimate the heart rate based on an estimate of the predicted level of exercise. This can be problematic for heart rate monitoring apparatuses which experiences a large amount of acceleration (e.g., in a sports setting), in which case the heart rate output may be either missing entirely or very inaccurate for a substantial amount of time during use.

Some heart rate monitoring apparatuses discard portions of the signal which are deemed too noisy by assessing signal quality (e.g., how clear spectral peaks are in the frequency domain). This can be helpful in removing noisy portions of the signal, but the data which is not discarded is not always reliable for heartbeat tracking. While such apparatuses can discard a portion of the signal that is too noisy, certain portions of the input signal exhibiting clear spectral peaks can still result in erroneous heartbeat readings because the spectral peaks could have been a result of periodic motion artifacts or other sources of artifacts affecting heart rate detection. For instance, a portion of the input signal degraded by motion artifacts but having clear spectral peaks could cause a heart rate tracking mechanism to lock onto a frequency corresponding to the motion artifact and not to the true heart rate.

Overview of an Improved Filtering Mechanism

The aforementioned problems of heart rate monitoring apparatuses stem from having a coarse mechanism for discarding input data, where, as used herein, the term “input data” (and variations thereof, such as e.g. “input signal”) refers to data from which a varying frequency, e.g. a heart rate, may be obtained. The present disclosure describes an improved filtering mechanism that alleviates some of the issues mentioned above. The improved filtering mechanism allows for a more nuanced processing of the raw input signal and can enable the input signal to be conditioned in such a way as to allow the tracker to track the heart rate better even when the signal was acquired in a noisy setting. By improving on the filtering mechanism, the heart rate monitoring apparatus can achieve more robust performance in a noisy environment. An improved filtering mechanism can increase the amount of the usable data of input signal and thereby increase the accuracy and consistency of heart rate output. Furthermore, the improved filtering mechanism can improve the accuracy of the tracking mechanism for tracking the heartbeat by way of providing a better and more usable input signal.

The improved filtering mechanism is based on recognition that, when a sensor, or multiple sensors, configured to generate an input signal from which the heart rate is to be tracked (such sensor or a plurality of sensors referred to in the following as a “heart rate sensor”) are moving (e.g. because a person wearing such heart rate monitoring apparatus is running), their measurements are affected by the movement in a predictable manner. Therefore, if the pattern of motion is known, then it may be possible to identify contributions to the input signal that are attributable to the motion of the heart rate sensor (i.e., motion-related artifact in the input signal) and filter those contributions out. The improved filtering mechanism then leverages an insight that, provided that a heart rate sensor is in relatively close proximity to an accelerometer so that both the accelerometer and the heart rate sensor experience the same motion, accelerometer measurements taken at the same time as the measurements by the heart rate sensor may be considered to accurately represent motion of the heart rate sensor when the input signal was acquired. In turn, accelerometer data related to the motion of the heart rate sensor may be used in reducing the amount of noise in the input signal generated by the sensor by identifying motion-related artifacts in the input signal. In particular, using STFTs of the input signal and of the accelerometer data and using, in an iterative manner, source models for a heartbeat and for motion sources allows creating a filter that reduces or eliminates motion-related artifacts from the input signal acquired by the heart rate sensor. As a result, identification/tracking of the heartbeat signal from a noisy sensor signal is improved.

The resulting filtering mechanism is able to better filter the input signal and improve the accuracy of heart rate tracking. The following passages describe in further detail how the improved filtering mechanism can be implemented and realized.

An Exemplary Improved Heart Rate Monitoring Apparatus and Method

FIG. 2 illustrate a system view of a heart rate monitoring apparatus, according to some embodiments of the disclosure. The system provides an arrangement of parts for implementing or enabling a method for tracking a varying frequency present in one or more input signals provided by one or more sensors in a noisy environment. Similar to FIG. 1, the apparatus includes a light source 102, an optical sensor 104. The light source can be a light emitting diode (LED), or any suitable component for emitting light. The light emitted by the light source 102 for measuring heart rate (e.g., blood volume) can be any suitable wavelength depending on the application. The apparatus can include a plurality of light sources emitting a range of wavelengths of light. The optical sensor 104 may be the same device as the light source 102, or the optical sensor 104 may be provided near the light source 102 to measure light near the optical sensor 104, e.g., to measure absorption of light emitted by the light source 102 in the skin to implement PPG. In addition, the apparatus includes an accelerometer 110 to measure acceleration/motion of the overall apparatus. Furthermore, the apparatus may, optionally, include other sensors 202 or other types of sensors, which can provide information to assist in filtering of the input signal and/or heart rate tracking. An integrated circuit 204 can be provided to drive the light source 102 and provide an analog front end 204 to receive signals provided by optical sensor 104, accelerometer 110, and other sensors 202. In some embodiments, the analog front end 204 can convert (if desired) analog input signals to data samples of the analog input signal. The analog front end can be communicate with a processor 206 to provide the data samples, which the processor 206 would process to track a varying frequency, e.g., the heartbeat.

In various embodiments, the processor 206 can include several special application specific parts or modules, electronic circuits, and/or programmable logic gates specially arranged for processing the data samples of the input signal to track the varying frequency. The processor 206 can be a digital signal processor provided with application specific components to track the varying frequency, and/or the processor can execute special instructions (stored on non-transitory computer readable-medium) for carrying out various methods of tracking the varying frequency as described herein. FIG. 3 illustrates an exemplary flow diagram of one such a method, e.g. implemented by the processor 206 shown in FIG. 2, for tracking a varying frequency present in one or more input signals provided by one or more sensors in a noisy environment, according to some embodiments of the disclosure. At a high level, the method includes a filter generation component 302, a signal conditioning component 304 (dependent on the filter generation component 302), and a tracking component 306 (dependent on the filter generation component 302 and/or the signal conditioning component 304). The method can continue back at the filter generation component 302 to process other data samples in the stream of data samples of the input signal.

Referring to both FIG. 2 and FIG. 3, in some embodiments, the parts of processor 206 can include one or more of the following: a filter generator 208, a signal conditioner 210, a tracker 212, and a reconstructor 216, e.g., to implement the method shown in FIG. 3.

The filter generator 208 implements functions related to the improved filtering mechanism (corresponding to filter generation component 302 of the method shown in FIG. 3) by using accelerometer data to generate a filter or a mask for filtering the input signal before providing the data samples to the tracker 212.

The signal conditioner 210 implement functions related to processing data samples of the input signal based on the decision(s) in the filter generator 208 to prepare the data samples for further processing by the tracker 212 (corresponding to signal conditioning component 304 of the method shown in FIG. 3). For instance, the signal conditioner 210 can filter data samples of the input signal a certain way (or apply a filter on the data samples), apply a mask to the data samples, attenuate certain data samples, modify the values of certain data samples, and/or select certain data samples from a particular sensor for further processing. The signal conditioning process can depend on the output(s) of the filter generator 208.

The tracker 212 implements functions related to tracking the varying frequency, e.g., the heartbeat, based on the output from the signal conditioner 210 (corresponding to tracking component 306 of the method shown in FIG. 3). In other words, the tracker monitors the incoming data samples (raw data or as provided by the signal conditioner 210) and attempts to determine the frequency of the varying frequency present in the one or more signals from the sensors. The output of the tracker 212, e.g., determined heart rate in beats per minute, can be provided to a user via output 214 (e.g., a speaker, a display, a haptic output device, etc.).

The reconstructor 216 can implement functions related to (re)constructing or synthesizing a time domain representation of the varying frequency, e.g., a heartbeat. Based on frequency information of the input signal, the reconstructor 216 can artificially generate a cleaner version of the input signal having the varying frequency (referred herein as the “reconstructed signal”). The reconstructed signal can be useful in many applications. For instance, the reconstructed signal can be provided to output 214 for display. The reconstructed signal can also be saved for later processing and/or viewing. Generally speaking, the reconstructed signal can be useful for users to visually and analytically assess the health of the living being with the irrelevant noise content removed. For instance, the reconstructed signal can assist healthcare professionals in assessing whether the living being has any underlying conditions relating to heart and arterial health. This reconstructed signal can be generated by first using the filter generator 208, the signal conditioner 210, and the tracker to track the varying frequency.

The filter generator 208, the signal conditioner 210, the tracker 212, and the reconstructor 216 can include means for performing their corresponding functions. Data and/or instructions for performing the functions can be stored and maintained in memory 218 (which can be a non-transitory computer-readable medium). In some embodiments, the filter generator 208 (corresponding to filter generation component 302 of the method shown in FIG. 3) can affect the processing performed in tracker 212 (corresponding to tracking component 306 of the method shown in FIG. 3). This feature is denoted by the arrow having the dotted line. The apparatus shown in FIG. 2 is merely an example of a heart rate apparatus, it is envisioned that other suitable arrangements can be provided to implement the improved method for filtering one or more input signals to track a varying frequency present in the input signals provided by heart rate sensors in a noisy environment.

Since embodiments of the present disclosure are based on evaluating time-dependent spectral characteristics such as the ones typically obtained using a Short Time Fourier Transform (STFT), basics of STFT as well as use of so-called “time-frequency bins” in context of signal source separation are now described.

Basics of STFT and Use of Time-Frequency Bins in Signal Source Separation

Processors described herein, such as e.g. the filter generator 208, may be configured to process data samples of an acquired signal (e.g. the input PPG signal or a signal from one of the accelerometer channels) to compute time-dependent spectral characteristics of the acquired signal. A characteristic could e.g. be a quantity indicative of a magnitude of the acquired signal. A characteristic is “spectral” in that it is computed for a particular frequency or a range of frequencies. A characteristic is “time-dependent” in that it may have different values at different times.

In an embodiment, such characteristics may be a Short Time Fourier Transform (STFT), computed as follows. An acquired signal is functionally divided into overlapping blocks, referred to herein as “frames.” For example, frames may be of a duration of 10240 milliseconds (ms) and be overlapping by e.g. 9600 ms. The portion of the acquired signal within a frame is then multiplied with a window function (i.e. a window function is applied to the frames), e.g. a Hann window, to smooth the edges. As is known in signal processing, and in particular in spectral analysis, the term “window function” (also known as tapering or apodization function) refers to a mathematical function that has values equal to or close to zero outside of a particular interval. The values outside the interval do not have to be identically zero, as long as the product of the window multiplied by its argument is square integrable, and, more specifically, that the function goes sufficiently rapidly toward zero. In typical applications, the window functions used are non-negative smooth “bell-shaped” curves, though rectangle, triangle, and other functions can be used. For instance, a function that is constant inside the interval and zero elsewhere is called a “rectangular window,” referring to the shape of its graphical representation. Next, a transformation function, such as e.g. Fast Fourier Transform (FFT), is applied transforming the waveform multiplied by the window function from a time domain to a frequency domain. As a result, a frequency decomposition of a portion of the acquired signal within each frame is obtained.

The frequency decomposition of all of the frames may be arranged in a matrix, referred to as an “STFT matrix” (in the simplest case, a two-dimensional array) where frames and frequency are indexed (in the following, frames are described to be indexed by “t” and frequencies are described to be indexed by “f”). Typically the frequency decomposition of each frame is arranged as a column of an STFT matrix (i.e. “t” is measured along a conventional x-axis), while the rows refer to different frequencies or frequency ranges (i.e. “f” is measured along a conventional y-axis). Each element of such an array, indexed by (f, t) comprises a complex value resulting from the application of the transformation function and is referred to herein as a “time-frequency bin” or simply “bin.” The term “bin” may be viewed as indicative of the fact that such a matrix may be considered as comprising a plurality of bins into which the signal's energy is distributed. In an embodiment, the bins may be considered to contain not complex values but nonnegative real quantities X(f,t) of the complex values, such quantities representing magnitudes of the STFT matrix bins, presented e.g. as an actual magnitude, a squared magnitude, or as a compressive transformation of a magnitude, such as a square root. In some implementations, the frequency bins above 3.5 Hz are not kept in the STFT matrix as such frequencies correspond to a heart rate of above 3.5×60=210 bpm, a reasonable upper bound for heart rate.

Time-frequency bins come into play in the improved filtering algorithm in that separation of a particular signal of interest, in context of this disclosure the signal of interest being a heartbeat signal (i.e. a signal generated by what is considered a specific “source” in the present disclosure) from the total signal acquired by a heart rate sensor (i.e., the total first signal) may be achieved by identifying which bins correspond to the signal of interest, i.e. when and at which frequencies the signal of interest is active. Once such bins are identified, the total acquired first signal may be masked by zeroing out the undesired time-frequency bins. Such an approach would be called a “hard mask.” Applying a so-called “soft mask” is also possible, the soft mask scaling the magnitude of each bin by some amount. Then an inverse transformation function (e.g. inverse STFT) may be applied to obtain the desired separated signal of interest in the time domain. Thus, masking in the frequency domain (i.e. in the domain of the transformation function) corresponds to applying a time-varying frequency-selective filter in the time domain.

Exemplary Implementation of an Improved Filtering Mechanism

Embodiments of the present invention are applicable both to cleaning up of an input signal (e.g. PPG signal) in the frequency domain as well as to tracking of the heart rate from the cleaned up signal. In some embodiments, once a PPG signal is cleaned up using the improved filtering mechanism described herein, any existing tracking algorithm may be used for tracking the heart rate from the cleaned up PPG signal (possibly after the cleaned up PPG signal is converted back into the time-domain). In other embodiments, heart rate may be obtained directly from an iterative mask estimation algorithm described herein (i.e. without the need to first convert the cleaned up PPG signal to the time-domain).

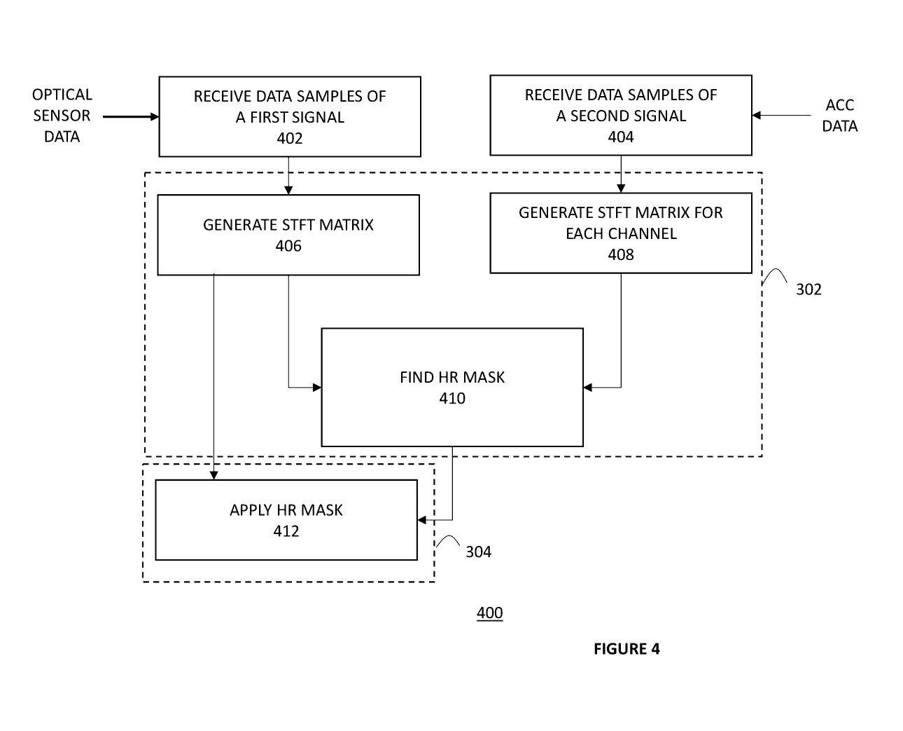

FIG. 4 illustrates an exemplary flow diagram of a more detailed method 400 for cleaning, or filtering, a heartbeat signal present in one or more input signals provided by one or more heart rate sensors in a noisy environment, according to some embodiments of the disclosure.

The improved filtering mechanism 400 may begin with receiving data samples of a first signal (i.e., an input signal such as e.g. a PPG signal) (step 402). The first signal can be generated by an optical sensor, and in some cases, the first signal is processed by an analog front end to produce (digital) data samples of the first signal.

The method 400 also includes receiving data samples of a second signal (step 404). The second signal can be generated by a device capable of detecting and quantifying motion of the heart rate sensors, such as e.g. an accelerometer. In order to quantify motion in all three orthogonal directions, the second signal preferably comprises three channels, one channel being for each accelerometer axis, detecting motion in that direction. Preferably, the second signal is acquired at the same time as the first signal and the two signals are processed synchronously, thus providing the closest overlap between the motion of the heart rate sensors and the data acquired by the sensors. Similar to the first signal, in some cases, the second signal is processed by an analog front end to produce (digital) data samples of the second signal.

The data samples of the first and second signals are received by the processor for filter generation, e.g. the filter generator 208.

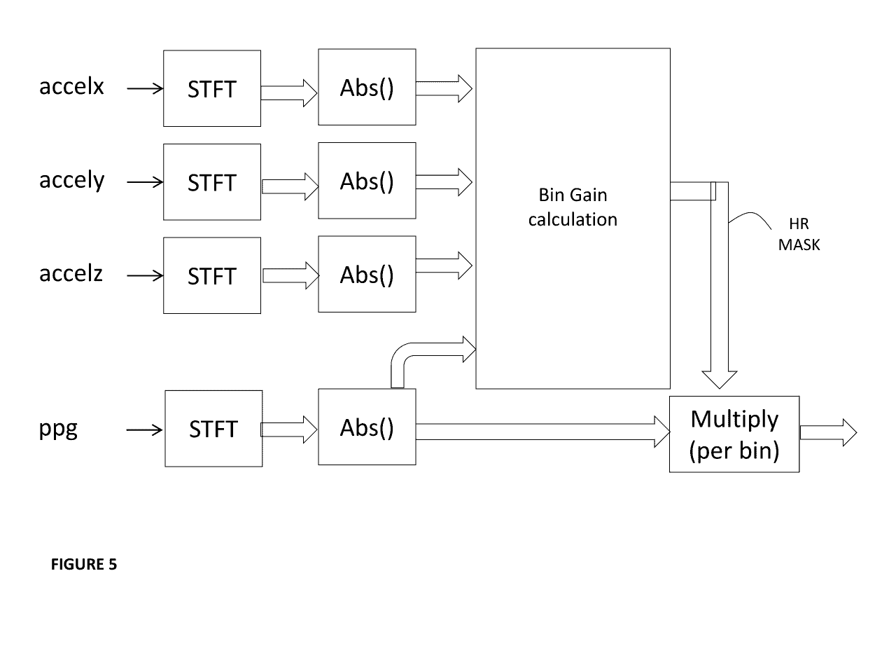

The filter generator 208 may then process the data samples of the first signal to compute time-dependent spectral characteristics of the acquired first signal (step 406). Similarly, the filter generator 208 may then process the data samples of the second signal to compute time-dependent spectral characteristics of the acquired second signal (step 408, possibly for each individual channel if the second signal contains data from multiple accelerometer channels). In an embodiment, such characteristics may be STFTs computed as described above. FIG. 5 illustrates generation of an STFT matrix for each of the PPG channel and three accelerometer channels, according to some embodiments of the disclosure. As shown in FIG. 5, each STFT matrix of the four matrices for each one of the channels may contain absolute values of magnitudes.

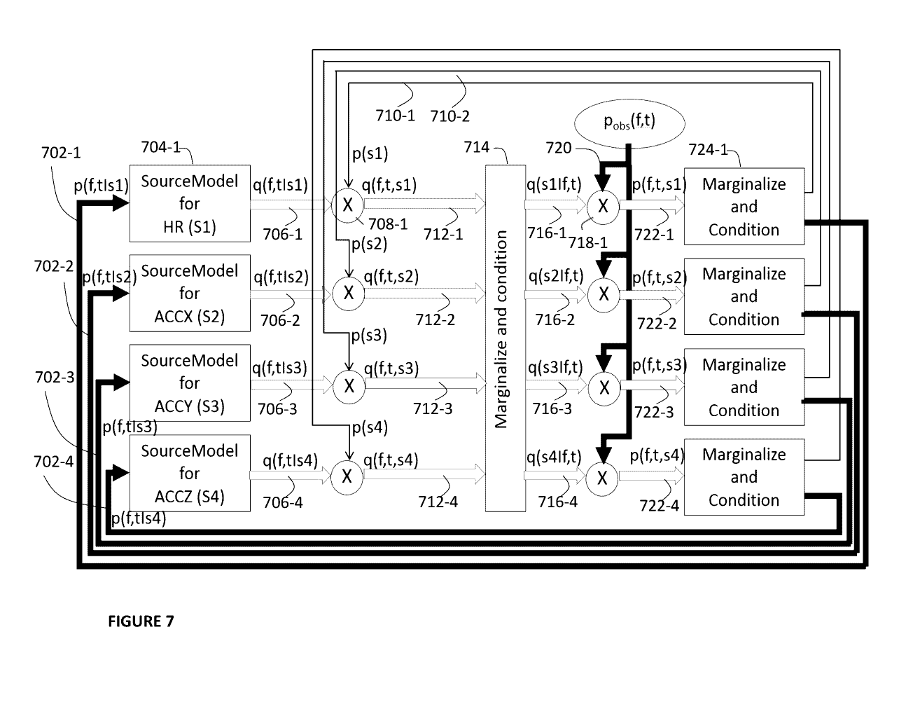

Embodiments of the present disclosure are based on recognition that PPG data, and therefore the STFT for the PPG channel, contains contribution from various signal sources (i.e. from various sources adding to the first signal detected by the heart rate sensor). In particular, PPG data contains contributions from the heartbeat, for which the present disclosure assumes a certain “source” referred to as heartbeat or heart rate source (“HR source”), as well as contributions from motion (i.e., motion-related artifacts that the filtering mechanism tries to reduce or eliminate), for which other “sources” are assumed. Each of the accelerometer channels is assumed to provide a reasonable approximation of a motion source in the direction of the accelerometer axis of the channel. Therefore, other “sources” considered in context of the present disclosure refer to accelerometer sources with one accelerometer source per accelerometer channel. The HR source is denoted herein as “s₁” or “HR (s₁)”, while the accelerometer sources for x-, y-, and z-axes of the accelerometer are denoted herein, respectively, as “ACCX (s₂)” (or “s₂”), “ACCY (s₃)” (or “s₃”), and “ACCZ (s₄)” (or “s₄”) (as illustrated e.g. in FIG. 7).

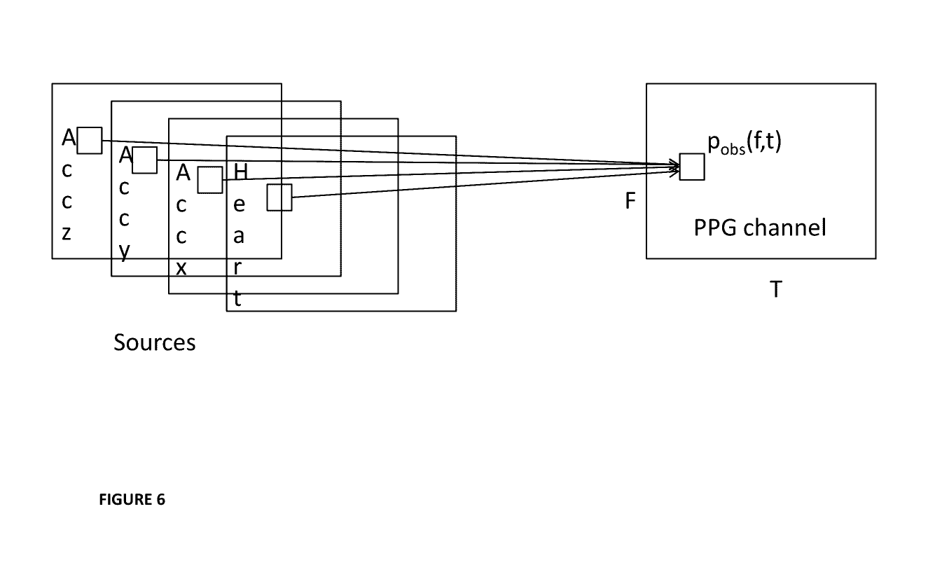

FIG. 6 illustrates contribution from individual sources to each time-frequency bin of a PPG channel, according to some embodiments of the present invention. As previously described, there are four channels that being measured, namely the PPG channel, and the three accelerometer channels. For each channel, the observed STFT magnitude is regarded as a probability distribution, scaled to sum to unity over (f,t), and referred to as “p_{obs}(f,t)”, “p_{accx}(f,t)^{”}, “p_{accy}(f,t)” and “p_{accz}(f,t)”, respectively. The STFT p_{obs}(f,t) is illustrated in FIG. 6. Energy in each (f,t) bin in PPG channel, i.e. “p_{obs}(f,t)”, consists of contributions from corresponding (f,t) bins from the four sources, namely the heart beat source and three motion sources, as also illustrated in FIG. 6.

Channels and sources are different concepts. The PPG channel is not representative of the pure heart beat source, but accelerometer channels happen to be good representatives of motion sources. In the p_{obs}(f,t) is illustrated in FIG. 6, contribution coefficients from each source to each (f,t) bin in PPG channel are assumed to sum to unity. The task of the filtering algorithm is then to find the mask isolating, or separating, the contribution from the heart rate source from the observed PPG signal, as explained below, during which estimates of the contribution coefficients from each source to the PPG channel are also computed as a by-product.

Turning back to FIG. 4, the four STFT matrices are then processed together, shown with a block “Bin Gain calculation” in order to determine individual contributions of each source (i.e., the HR source, ACCX, ACCY, and ACCZ sources) to the PPG signal (i.e. to the PPG channel) (step 410 in FIG. 4). Therefore the filtering mechanism described herein may be considered a “source separation problem.” The bin gains are collectively referred to as masks. In an embodiment, individual contributions of each source to the PPG signal are estimated as the product of the STFT of the PPG and the mask corresponding to a particular source.

As described below, as a by-product of computation of the HR mask, masks for the accelerometer sources are obtained as well, which are used as intermediate inputs for the improved filtering mechanism described herein.

The computed HR mask is then applied to the PPG channel data to substantially reduce contributions to the PPG channel from all sources other than the HR source, as shown with step 412 of FIG. 4. In an embodiment, application of mask may include point-wise multiplication of the STFT matrix for the PPG channel with the HR mask matrix, as illustrated with a box “Multiply (per bin)” in FIG. 5.

Applying the HR mask to the PPG signal reduces contributions in the PPG signal that are attributable to the motion of the heart rate sensor (i.e, motion-related artifacts), thereby generating a filtered first signal where the amount of noise is reduced by eliminating or at least reducing noise due to the motion of the heart rate sensor. Such a filtered first signal may then be provided to the back-end heart rate estimation using any of the known tracking algorithms for actually tracking the heart rate (not shown in FIG. 4). To that end, the filtered first signal may either be converted back to a time-domain (e.g. by applying inverse STFT) or could stay in the frequency domain for heart rate tracking.

In FIG. 4, steps 406-412 may be considered to represent the filter generation 302 described in FIG. 3, as indicated with a dashed box labeled “302” around these steps in FIG. 4. On the other hand, step 412 may be considered to represent the signal conditioning 304 described in FIG. 3, as indicated with a dashed box labeled “304” around this step in FIG. 4.

Although not shown in FIG. 4, it should be noted that prior to or as a part of processing of the PPG input (first signal) and accelerometer data (second signal), in some embodiments, a filter may be applied to one or both of these signals to filter out contributions in each of these signals that cannot be attributable to the heartbeat. Such a filter would be configured to filter out components outside of an expected range of frequencies representative of the frequency of interest (i.e., a heartbeat). Typically, a heart rate is between 0.5 Hertz to 3.5 Hertz (in some cases it can be as high as 4 or 5 Hertz). The filtering using such filters could alternatively be performed at later points in time, but preferably before the step of tracking the heart rate. In an embodiment, such a filter can be incorporated with a signal conditioning process by processing the data samples with a filter to substantially attenuate, within the PPG signal, signal content outside of a reasonable frequency band of interest corresponding to the heartbeat signal (or apply a masking process to achieve a similar effect) before extracting the heart rate information of the PPG signal. Such a filter could be implemented e.g. as a band-pass filter (e.g., passing signals in the bandwidth from 0.5-3.5 Hertz, 0.5-4 Hertz, 0-4.5 Hertz, or similar variant thereof) or as a low-pass filter (e.g., passing signals in a bandwidth from 0-3.5 Hertz, 0-4 Hertz, 0-4.5 Hertz or similar variant thereof). The type of filter used to attenuate signals outside of the reasonable frequency band of interest can vary depending on the application. Furthermore, the reasonable frequency band of interest can vary depending on the application. In one example, the reasonable frequency band of interest includes a frequency band of 0.5 Hertz to 3.5 Hertz (or includes frequencies between 0.5 Hertz to 3.5 Hertz), which is suitable for keeping frequency content that is more likely to be associated with a heartbeat.

Steps 410-412 illustrated in FIG. 4 are now described in greater detail with reference to an iterative frequency-domain mask estimation algorithm illustrated in FIG. 7.

Iterative Mask Estimation Mechanism

FIG. 7 illustrates an example of iterative mask estimation mechanism, according to some embodiments of the disclosure.

As shown in FIG. 7, all quantities may be processed as if they are probability distributions over one or more of the following random variables: s (for sources), f (for frequency index), and t (for time index, i.e. index of a time frame, the time frame representing a certain range of times). In the following, probabilities “q” and “p” refer to, respectively, before and after applying masks for each source to the measured PPG data (which may be referred to as “folding in the observed PPG data”) at each step of the iteration.

The following notation is used:
q(f,t,s) or p(f,t,s)=The probability of a unity energy having come from source s and ended up in the (f,t) bin,
q(f,tls) or p(f,tls)=Given a source s, what is the distribution of its energy over time frequency bins (f,t),
q(s) or p(s)=The total energy contribution from source s to PPG channel (sum over all bins),
q(slf,t)=(MASK) The relative proportion of a source s present in the given time-freq bin (f,t) of PPG,
p_{obs}(f,t)=The (normalized) STFT of the observed PPG channel featuring both heart rate and the motion artifact contributions (fixed throughout the iterations), and
p_{accx}(f,t)=The (normalized) STFT of the observed accx channel (similar notation is used for other accelerometer channels, i.e. channels accy and accz).

The heart rate source is estimated as p_{obs}(f,t) *MASK1 (where MASK1 is the HR mask) at each iteration, which needs to be scaled with p(s₁) so that it is a valid probability distribution. Other sources are estimated by applying their corresponding masks and conditioning with corresponding p(s) values.

In FIG. 7, the thick arrows represent matrices (2D probability distributions, e.g. STFTs), while the thin arrows represent scalar values that scale the matrices (i.e. that scale the STFTs). Furthermore, the loop formed by the arrows 706, 712,716, 722 leading to the computation of quantities carried by arrows 702 and 710 is referred to as an “outer loop” of the iterative source separation algorithm, while any loops that may take place within the SourceModel blocks are referred to as “inner loop.”

Each source model of the four source models (704-1 through 704-4) illustrated on the left side of FIG. 7 receives a respective current STFT estimate for the source as input, shown as inputs 702-1 through 702-4. Each source model module applies the received current STFT estimate to its source model (i.e. what the model “thinks” the STFT for the source “should” look like) and provide as an output an updated STFT for each source, shown as updated STFTs 706-1 through 706-4.

Each updated STFT is then scaled, shown in FIG. 7 as multiplication points 708-1 through 708-4, with a respective current estimate for total contribution from each source to the PPG signal, shown as estimates 710-1 through 710-4. As a result, a weighted STFT estimate is obtained for each source, as shown with 712-1 through 712-4. All weighted STFT estimates are then provided to a thin block “Marginalize and condition” 714.

The thin “Marginalize and Condition” box in the middle of the slide takes as input four matrices, namely q(f, t, s=s₁), q(f, t, s=s₂), q(f, t, s=s₃), q(f, t, s=s₄). Each of these is in fact a slice of the three dimensional tensor q(f, t, s), each layer corresponding to one source. This probability distribution q(f, t, s) is called the “joint distribution” of the random variables f, t, s. The “marginalize and condition” box outputs the mask q(s=sᵢ|f, t), i=1, 2, 3, 4 by first marginalizing out s from the joint distribution to obtain q(f, t):

$$q ( f , t ) = {\sum }_{i = 1}^{4} q ( f , t , {s}_{i} ) \qquad ( 1 )$$

and then computing the conditional distinction

$$q ( s = {s}_{i} \vert f , t ) = \frac{q ( f , t , {s}_{i} )}{q ( f , t )} \qquad ( 2 )$$

Each of the outputs q(s=sᵢ|f, t) is simply one layer of this computed conditional distribution corresponding to the current estimate of the mask for that source.

Each of the four “Marginalize and Condition” blocks at the right part of the slide does a similar thing. These take as input p(f, t, s=s₁), p(f, t, s=s₂), p(f, t, s=s₃), p(f, t, s=s₄), respectively. Then each computes the marginal, which is one of the two outputs of this block

$$p ( s = {s}_{i} ) = {\sum }_{f = 1}^{F} {\sum }_{t = 1}^{T} p ( f , t , s = {s}_{i} ) \qquad ( 3 )$$

for i=1, 2, 3, 4. The second output is obtained by computing the conditional distribution

$$p ( f , t \vert s = {s}_{i} ) = \frac{p ( f , t , s = {s}_{i} )}{p ( s = {s}_{i} )} \qquad ( 4 )$$

As the foregoing description illustrates, the outcomes of the block 714 are masks for the individual sources, shown as masks 716-1 through 716-4 in FIG. 7. At multiplication blocks 718-1 through 718-4, each one of these masks is multiplied with the STFT of the PPG channel (also provided as an input to the blocks 718, as can be seen in FIG. 7 with an arrow 720). Since each one of the masks 716 is a matrix and the STFT 720 of the PPG signal is a matrix, multiplication at blocks 718 is a point-wise multiplication, where a value in each bin of a mask matrix is multiplied with a value in a corresponding bin of the PPG STFT.

Multiplication of mask for source sᵢ with PPG channel STFT results in an STFT for that source, shown as output STFTs 722-1 through 722-4. Such an STFT for each source is then provided to Marginalize and Condition block 724-1 through 724-4, the functionality of which was explained above.

The first output of each respective Marginalize and Condition block 724 (i.e., the scalar output calculated in accordance with equation (3) above) is proved as respective input 710 to the respective multiplication block 708, as shown in FIG. 7. As also shown, the second output of each respective Marginalize and Condition block 724 (i.e., the matrix output calculated in accordance with equation (4) above) is proved as respective input 702 to the respective source model 704. Each source model then updates itself based on the input 702, as needed, and the cycle continues.

FIG. 7 illustrates four different marginalize and condition blocks 724 but only one block 714. One reason for such illustration is that the block 714 operates on all four inputs 712 to compute the quantity q(f, t) by marginalization, using summation over all sources sᵢ, where i=1, 2, 3, 4 in accordance with equation (1) above. On the other hand, for blocks 724, each input is independent of the other sources because the summation is only over the (f, t) indices and not s, so the functionality of the blocks 724 could be separate, e.g. in order to benefit from parallel processing. A hardware implementation of the approach shown in FIG. 7 may benefit from this distinction.

Source Models

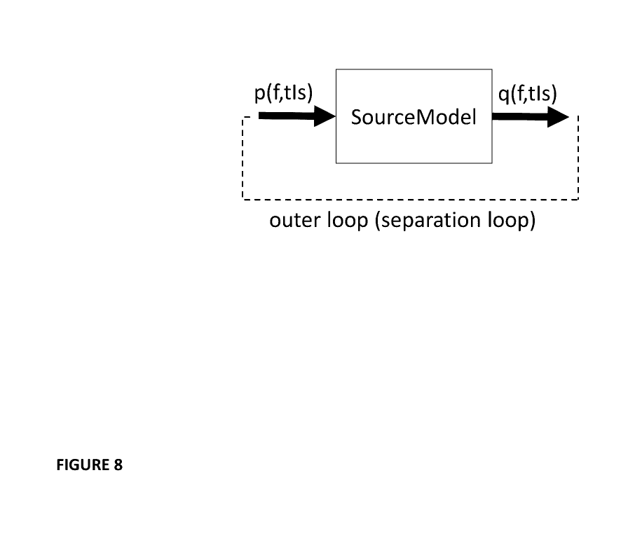

Source Models for each source are “modules” (shown as blocks 704 in FIG. 7) that output an estimate of STFT for that source, or in the developed probabilistic notation q(f,tls), based on an input p(f,tls) provided from the main loop. This is schematically illustrated in FIG. 8.

Source Models can take several forms based on what they do, and/or what they don't do, with the input p(f,tls). For the Source Models of the accelerometer sources, we have “an estimate” of what each accelerometer channel (and, therefore, source) looks like, so there is an initial learning step that includes learning the accelerometer sources before starting source separation illustrated in FIG. 7. The HR source is not learned because a clean measurement of it, like the ones for the accelerometer sources, is typically not available.

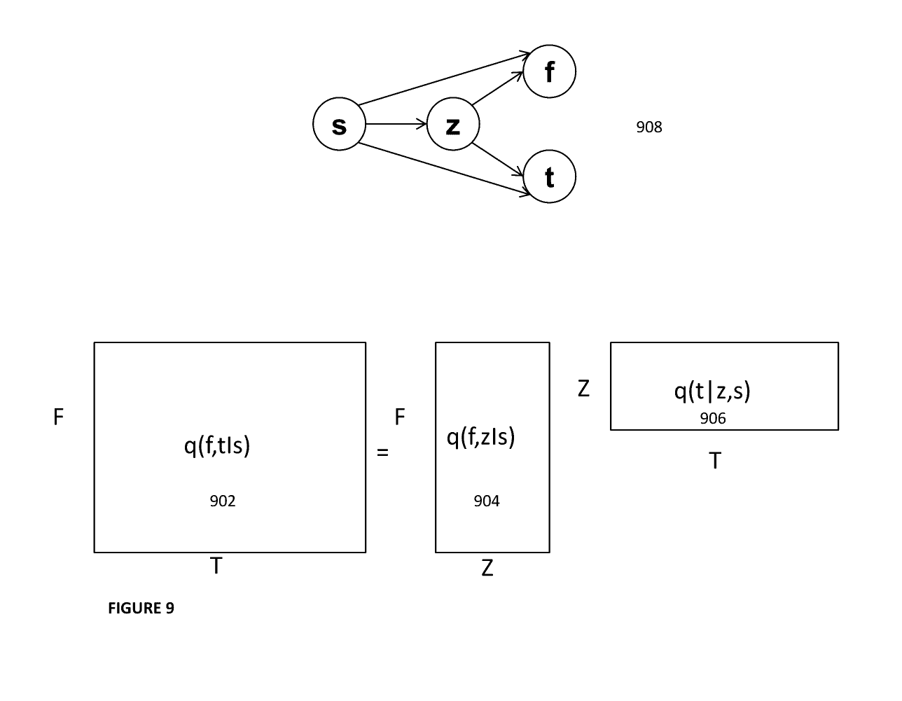

In some embodiments, a Non-negative Matrix Factorization (NMF) source model may be used, as illustrated in FIG. 9. Such a source model could be used to model both the HR source and ACC sources. An NMF approximates the STFT (702, 706 in FIG. 7), shown as a matrix 902 in FIG. 9 (variable T is the number of columns and variable F is the number of rows in the STFTs described here), as a product of a “tall” matrix 904 (variable Z is the number of dictionary elements), where columns are dictionary elements, and a “fat” matrix 906, where columns are time activations, with an inner dimension usually much lower than the actual rank, therefore providing a low rank approximation (i.e., Z is on the order less than the minimum of F and T, hence lower rank). This is equivalent to a graphical model 908 shown in FIG. 9, where z is the dictionary element random variable.

For a given source s:

q(f,t|s)=Σ_{z}q(f,z|s)·q(t|z,s)

NMF source model owns and maintains a set of dictionary elements q(f,zls) and the corresponding time activations q(tlz,s). During training and separation stages, these are occasionally updated with the incoming information p(f,tls).

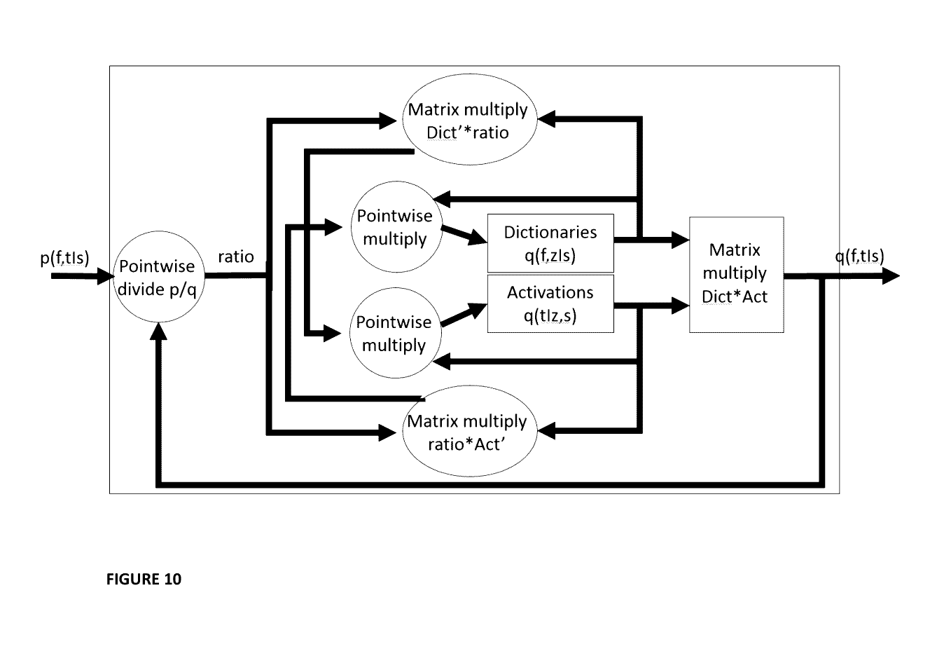

FIG. 10 illustrates the internal process flow of an NMF source model during training and separation, according to some embodiments of the disclosure. FIG. 10 illustrates updating parameters for dictionaries and/or activations. It is also possible to keep one of them constant and only update the other one (e.g. keep dictionaries constant and only update activations). In FIG. 10, “Act” denotes a transpose of Act, which is the activations matrix 906 shown in FIG. 9.

In an embodiment, one update cycle may be used for the dictionaries and activations such that q(f,tls) gets closer to p(f,tls) (in terms of KL divergence) while maintaining low rank NMF factorability.

For training, input p(f,tls) is fixed to the measured accelerometer channel, e.g. p_{accx}(f,t). Typically 10-20 iterations are enough to learn the dictionaries and activations, which could be initialized randomly at first. After the training is complete, the learned activations and dictionaries for accelerometer sources are kept for separation stage and used as source models in the iterative mask estimation mechanism, e.g. as source models 704-2 through 704-4 shown in FIG. 7.

For separation, input p(f,tls)=masked p_{obs}(f,t) with conditioning applied. In an embodiment, only one update per separation (outer) loop iteration is applied to time activations (dictionaries are fixed as learned from training).

The operation in box “Matrix multiply” shown in FIG. 10 is as follows:

$$q ( f , t \vert s ) = {\sum }_{z = 1}^{Z} q ( f , z \vert s ) q ( t \vert z , s ) \qquad ( 5 )$$

The summation over all the values of the random variable z is why this is a marginalization over z and the operation could be called “multiply and marginalize”, in line with the definition of matrix multiplication with the inner index being z as shown in FIG. 9.

In various embodiments, other source models or a combination of source models may be used and/or source models already learned during training could be further updated during the separation stage. For example, NMF source models may be used for motion sources, but the dictionaries could also be updated during the separation stage.

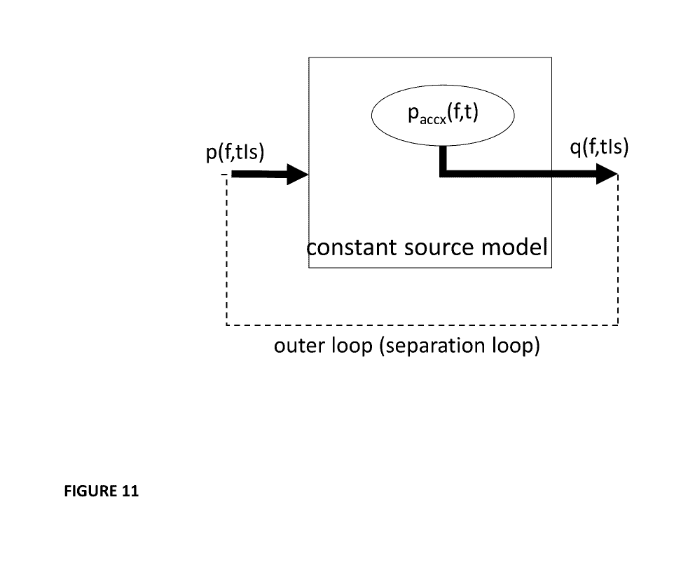

In some embodiments, constant source models may be used for one or more of the motion sources. Such a model is illustrated in FIG. 11, for one accelerometer source (other sources could use a similar model or could use a different, e.g. NMF, model). In a constant source model, the learning stage trivially sets q(f,tls)=p(f,tls) with no updates or iterations, where, e.g., p(f,tls)=p_{accx}(f,t) since the input is typically fixed to an observed channel during the training stage. During separation, it always reports this constant value no matter what the input p(f,tls) is, thus strictly enforcing the motion sources to be equal to the measured accelerometer channels. The generated mask for these motion sources will not strictly agree with this enforcement because of the effect of the outer loop that also takes into account the contribution of the HR source to the PPG channel, therefore improvement on the HR source can still be expected with increased iterations.

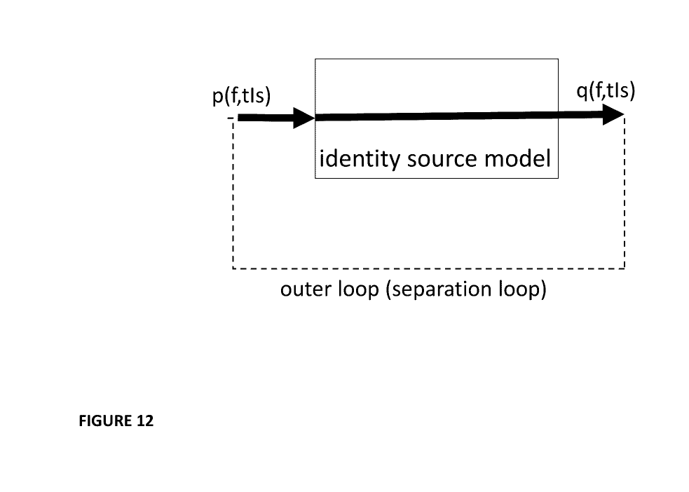

While a constant source model could be used for motion sources, it's less desirable to use it for HR source because a clean measurement of source (HR) is typically not available. For the HR source, an identity source model may be used, as illustrated in FIG. 12. In this model, the “training stage” may be considered as not being a training stage but just a random initialization for q(f,tls) so that the model has something to output in the first iteration of the outer loop of the iterative mask estimation. During separation, an identity source model behaves as an identity function that shorts its input to the output with no changes on it. Such a source model is not suitable for motion sources as it will quickly “forget” the valuable prior information about the motion sources.

Using an NMF source model for all sources has been shown mathematically to decrease KL divergence at each step of the inner (training) loop and the outer (separation) loop. The combination of source models where an identity source model is used for HR source and constant source model is used for the motion sources has a trivial solution and therefore is not preferred. Other combinations of source models are also possible and are within the scope of the present disclosure, e.g. an NMF source model for the HR source and a constant source model for each of the motion sources.

Another example, a one-peak source model may be used for the HR source model, which is a parametric source model that fits a parametric form to each column of the estimate of an STFT for the HR source (i.e., p(f,t|s₁)). More specifically, the DFT of the analysis window (e.g. Hann) may be shifted across all frequencies and compared to each column of the STFT for the HR source. The comparison may be done using a dot product and the frequency at which the largest dot product occurs may be reported as the estimated heart rate for that iteration. Therefore, this source model is able to yield estimated heart rate at each iteration as a by-product, thus eliminating the need to use a separate tracking algorithm. In an embodiments, costs may be placed on variations of HR from one frame to another to enforce HR smoothness.

Since the one-peak source model allows only one peak in each column of the STFT for the HR source, there may remain energy peaks in the PPG that are not explained by the motion sources either. Another source, referred to herein as a “dustbin source” may be used to collect all unexplained energy of the PPG channel.

In an embodiment, one-peak source model may be used for the HR source, constant source models for the accelerometer sources, and an NMF source model for dustbin.

Iterative Mask Estimation Mechanism with Color Information

NMF models described above are referred to as “NMF” (and not “NTF”) because there was no third dimension, so there was only a matrix to factorize instead of a tensor:

$${P}_{obs} ( f , t ) = {\sum }_{5} q ( s ) {\sum }_{z} {\underbrace{q ( \underline{f , zls} ) q ( \underline{tlz , s} )}}_{NMF source model}$$

In an embodiment, p_{obs} may be extended to a third dimension by including color as the third dimension, and its factorization also becomes NTF:

$${P}_{obs} ( \underline{f , t , c} ) = {\sum }_{5} q ( s ) q ( \underline{cls} ) {\sum }_{z} {\underbrace{q ( \underline{f , zls} ) q ( \underline{tlz , s} )}}_{NMF source model}$$

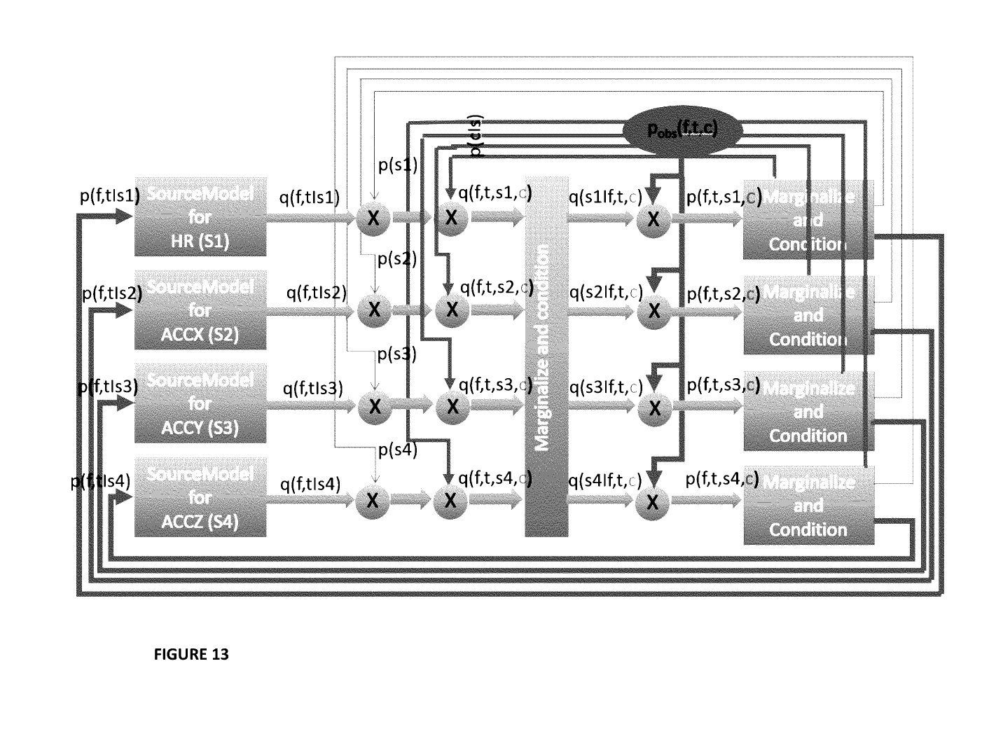

In an embodiment, the PPG channel may be measured with green light. In some cases, it may also be measured with infrared or red light as well. For including the information coming from other colored LEDs, the basic iterative mask estimation algorithm described above is extended to include this extra information, as shown in FIG. 13.

FIG. 13 illustrates an example of iterative mask estimation mechanism using color information, according to some embodiments of the disclosure. Notation and explanations of the elements and activities depicted in FIG. 13 are similar to those described for FIG. 7, except that color information is used (denoted as “c”), which adds a few steps illustrated in FIG. 13. Furthermore, the thinnest arrows “carry” scalar values or coefficients, slightly thicker arrows correspond to 1D arrays or vectors and the thickest arrows correspond to at least 2D arrays such as matrices and tensors.

With such an approach, a three dimensional tensor, p_{obs}(f, t, c), is involved, instead of p_{obs} (f, t) described for FIG. 7. Each layer of this three dimensional tensor corresponds to the STFT of the corresponding color's PPG channel normalized to sum to unity and multiplied with (1/C), where C is the number of colors available such that the whole tensor is a probability distribution that sums to unity. For example if green and infrared channels are used, a PPG signal is measured using each color channel (i.e. then references are made to multiple PPG channels, one PPG channel per color), an STFT for each PPG channel is computed, which is still a matrix, each one is scaled to sum to unity, then multiplied by ½, and these matrices are stacked to have a three dimensional p_{obs} (f, t, c). FIGS. 7 and 13 differ in the dimensionality of p_{obs} in its argument.

Due to the use of color information, FIG. 13 also has some additional features when compared to FIG. 7.

The first one is that each q(f, t, s=sᵢ) branch gets multiplied with the probability distribution of color conditioned on each source: p(c|s=sᵢ). In this case, the “marginalize and condition” block is doing slightly more than equation (1) and (2). The first step is to compute (marginalization part of it)

$$q ( f , t , c ) = {\sum }_{s = 1}^{4} q ( f , t , s , c ) \qquad ( 6 )$$

and then (computing the conditional distribution)

$$q ( s \vert f , t , c ) = \frac{q ( f , t , s , c )}{q ( f , t , c )} \qquad ( 7 )$$

This creates a mask for each source, which is in turn multiplied with p_{obs}(f, t, c) to obtain p(f, t, c, s=sᵢ), i=1, 2, 3, 4. Finally, each of the last four “Marginalize and Condition” blocks computes three sets of outputs, namely p(s=sᵢ), p(c/s=sᵢ) and p(f, t/s=sᵢ) for i=1, 2, 3, 4.

To do this, it computes

$$\begin{matrix}p ( f , t , s = {s}_{i} ) = {\sum }_{c = 1}^{C} p ( f , t , c , s = {s}_{i} ) , & ( 8 ) \\ p ( c , s = {s}_{i} ) = {\sum }_{f = 1}^{F} {\sum }_{t = 1}^{T} p ( f , t , c , s = {s}_{i} ) , \\ and & ( 9 ) \\ p ( s = {s}_{i} ) = {\sum }_{c = 1}^{C} p ( c , s = {s}_{i} ) , & ( 10 )\end{matrix}$$

for i=1, 2, 3, 4. All of these “marginalization” operations. Then, the outputs are computed as (these are conditionings)

$$\begin{matrix}p ( c \vert s = {s}_{i} ) = \frac{p ( c , s = {s}_{i} )}{p ( s = {s}_{i} )} \\ and & ( 11 ) \\ p ( f , t \vert s = {s}_{i} ) = \frac{p ( f , t , s = {s}_{i} )}{p ( s = {s}_{i} )} & ( 12 )\end{matrix}$$

The output p(s=sᵢ) were already computed above.

In the embodiment of FIG. 13, since none of the marginalizations are performed over sources s, all marginalization blocks can be separated as described above for FIG. 7, thus providing for further flexibility in terms of possible parallel processing.

Advantages, Variations and Implementations

Some advantages realized by the application of the improved filtering method described herein include systematic computation of soft gains trying to progressively minimize the Kullback-Leibler (KL) divergence between a signal obtained by incorporating the observed signal and a signal that fits a reasonable signal model, possibility of extending the approach to incorporate external information such as different color light easily, possibility to mix and match different source models and any number of them, flexibility to include other interference sources (e.g., dustbin source referring to all other sources of contributions to the PPG signal besides the HR source and the ACC sources, i.e. a source modelling all unexplained energies in the bins of the PPG STFT), ability to use non-calibrated accelerometer data (scaling and offset), and the method not being limited to sinusoid interference signals only since gain computations don't rely on peak values.

While examples described herein are based on using motion data from three accelerometer channels, the improved filtering method may also be implemented in an analogous manner by using only one or two of the accelerometer channels, or by using some combination of two or more accelerometer channels.

While many examples described herein are described in relation to a frequency representative of a heartbeat, it is envisioned that the method can be applicable in other scenarios for filtering an input signal used for tracking other types of slowly varying frequencies (e.g., phenomena or events which has a frequency that does not change or jump abruptly) as well as tracking frequencies which are not necessarily slowly varying. Furthermore, while the examples herein are described with one or more input signals provided by one or more optical sensors, it is envisioned that the method can be used to filter the input signals generated by other types of sensors, including but not limited to: optical sensor, audio sensor, capacitive sensor, magnetic sensor, chemical sensor, humidity sensor, moisture sensor, pressure sensor, and biosensor.

Furthermore, more than one optical sensor may be used and data obtained therefrom may be filtered according to the improved filtering method described above. Some considerations for using more than one optical sensors are described in the following section.

Using More than One Optical Sensor

The wavelengths used for measuring input signals for PPG can span wavelengths from blue to infrared. In classic applications, LEDs of two colors—often 660 nm and 940 nm—are used for measuring blood oxygen saturation. These devices are in large volume production and are readily available. In yet another application, a simple single-color LED—say at 940 nm, may be used to measure heart rate by measuring the periodic variation in a return signal. In some cases, a green LED is used to pick up variation in absorption caused by blood flow on the wrist.

Different wavelengths of light reflects differently from skin (due to the pigmentation and wrinkles, and other features of the skin) and different optical sensors tend to behave differently in the presence of motion when sensing light reflected from skin. Based on this insight, it is possible to infer information about presence of motion and/or the quality of an input signal. It is also possible to improve the data samples to be processed by the tracker based on the insight. Multiple light sources having different wavelengths can be used (e.g., a red LED and a green LED). For instance, by sensing these light sources and examining differences between the input signals of optical sensors for detecting light having respective wavelengths, or different portions of a spectrum of an input signal from a wideband optical sensor, it is possible to infer whether certain data samples of the input signal is likely to have been affected by motion or some other artifact.

Broadly speaking, an internally consistent model can be provided if different characteristics and behavior of different types of optical sensors under various conditions (or in general) are known. Based on the internally consistent model, information about the signal or the environment of the sensors can be inferred. The inference can assist filter generators in assessing whether certain portions of the data samples should be removed. The inference can also assist signal conditioning to specify how the data samples should be processed to improve tracking. This can include filtering the signal a certain way. The inference can also, in some cases, signal to the tracker to perform tracking differently.

In some instances, the use of multiple optical sensors can improve tracking by removing or subtracting common global characteristics between optical sensors to better track the varying frequency. In some cases, the internally consistent model may prescribe that the tracked varying frequency (e.g., slowly varying frequency such as the tracked heart rate) should be substantially the same for a plurality of sensors (e.g., the red LED should measure the same heart rate as the green LED).

### Selected Examples

Example 1 provides a method comprising at least some of steps as illustrated in FIGS. 4 and/or 7.

Example 2 provides the method according to Example 1, further comprising processing the data samples of the second signal with a pre-processing filter to substantially attenuate signal content outside of a reasonable frequency band of interest corresponding to the heartbeat, and/or processing the data samples of the first signal with a pre-processing filter to substantially attenuate signal content outside of the reasonable frequency band of interest corresponding to the heartbeat.

Example 3 provides the method according to Example 2, wherein the pre-processing filter is a low-pass filter or a band-pass filter; and the reasonable frequency band of interest comprises frequencies between 0.5 Hertz to 3.5 Hertz.

Example 4 provides the method according to Example 1, further comprising applying a filter or mask to or removing a portion of the data samples of the first signal indicative of a saturation condition of the one or more sensors.

Example 5 provides the method according to Example 1, further comprising processing the filtered first signal to track the heart beat signal.

Example 6 provides the method according to Example 5, wherein processing the filtered first signal comprises generating a time-frequency representation of the filtered first signal; and tracking one or more contours present in the time-frequency representation to track the heartbeat signal.

Example 7 provides the method according to Example 1, wherein the one or more sensors include one or more of the following: optical sensor, audio sensor, capacitive sensor, magnetic sensor, chemical sensor, humidity sensor, moisture sensor, pressure sensor, and biosensor.

Example 8 provides an apparatus comprising at least one memory configured to store computer executable instructions, and at least one processor coupled to the at least one memory and configured, when executing the instructions carry out a method according to any one of Examples 1-7.

Example 9 provides a non-transitory computer readable storage medium storing software code portions configured for, when executed on a processor, carrying out a method according to any one of Examples 1-7.

Example 10 provides an apparatus comprising means for performing a method according to any one of Examples 1-7.

Example 11 provides a computer-implemented method for assisting separation of a heartbeat signal present in a first signal generated by a heartbeat sensor in a noisy environment. In context of a “heartbeat sensor”, the term “heartbeat” is merely used to differentiate sensors generating PPG data from other sensors, such as e.g. accelerometers; the term “heartbeat signal” refers to a contribution of a heartbeat source to the first signal generated by the heartbeat sensor. The “heartbeat signal” refers to a signal indicative of (i.e. being representative of) the heartbeat of the subject being measured. The method includes processing the first signal to compute a first time-frequency (TF) matrix of F×T dimensions where T is an integer indicating a number of time frames t and F is an integer indicating a number of frequency ranges f and, the first TF matrix including a time-frequency-domain representation (p_{obs}(f,t)) of the first signal; processing a second signal to compute a second TF matrix of T×F dimensions, the second TF matrix including a time-frequency-domain representation (p_{accx}(f,t)) of the second signal, the second signal indicative of a motion of the heartbeat sensor with respect to a first direction (x); initializing a first source model configured to generate an estimate of a TF matrix (q(f,t|s₁)) for a first source (s₁), the first source representing a source of the heartbeat signal; initializing a second source model configured to generate an estimate of a TF matrix (q(f,t|s₂)) for a second source (s₂), the second source representing a source of a contribution to the first signal due to the motion of the heartbeat sensor with respect to the first direction, where the second source model is initialized based on the time-frequency-domain representation (p_{accx}(f,t)) of the second signal; performing a plurality of iterations of modifying one or more parameters of the first source model and/or one or more parameters of the second source model based on the time-frequency-domain representation of the first signal; and, following the plurality of iterations, computing an estimate of the heartbeat signal based on the one or more parameters of the first source model and/or the one or more parameters of the second source model.

Example 12 provides the method according to Example 11, where each iteration of the plurality of iterations includes using the first source model to generate an estimate of a time-frequency-domain representation (q(f,t|s₁)) for the first source (i.e. an estimate of a time-frequency-domain representation, in the form of e.g. a TF matrix, for a contribution to the first signal that is attributed to the first source), using the second source model to generate an estimate of a time-frequency-domain representation (q(f,t|s₂)) for the second source (i.e. an estimate of a frequency-domain representation, in the form of e.g. a TF matrix, for a contribution to the first signal that is attributed to the second source), and based on the estimate of the time-frequency-domain representation for the first source and the estimate of the time-frequency-domain representation for the second source, computing a first mask function q(s₁|f,t) for separating the heartbeat signal from the first signal and computing a second mask function q(s₂|f,t) for separating, from the first signal, the contribution due to motion of the heartbeat sensor with respect to the first direction.

Example 13 provides the method according to Example 12, where said each iteration of the plurality of iterations further includes applying the first mask function to the time-frequency-domain representation of the first signal to generate an updated estimate of a time-frequency-domain representation (p(f,t|s₁)) for the first source, applying the second mask function to the time-frequency-domain representation of the first signal to generate an updated estimate of a frequency-domain representation for the second source (s2), applying the updated estimate of the time-frequency-domain representation for the first source to update one or more parameters of the first source model, and applying the updated estimate of the frequency-domain representation for the second source to update one or more parameters of the second source model.

Example 14 provides the method according to Example 13, where applying the first mask function includes performing a point-wise multiplication of the first mask function and the time-frequency-domain representation of the first signal, and applying the second mask function includes performing a point-wise multiplication of the second mask function and the time-frequency-domain representation of the first signal.

Example 15 provides the method according to Example 11, where the first source model includes a Non-negative Tensor Factorization (NTF) source model or a one-peak source model, and/or the second source model includes the NTF source model or a constant source model.

Example 16 provides the method according to Example 11, further including processing a third signal to compute a third TF matrix of T×F dimensions, the third TF matrix including a time-frequency-domain representation (p_{accy}(f,t)) of the third signal, the third signal indicative of a motion of the heartbeat sensor with respect to a second direction (y); and initializing a third source model configured to generate an estimate of a TF matrix (q(f,t|s3)) for a third source (s3), the third source representing a source of a contribution to the first signal due to the motion of the heartbeat sensor with respect to the second direction, where the third source model is initialized based on the time-frequency-domain representation (p_{accy}(f,t)) of the third signal, where the estimate of the heartbeat signal is computed based on the one or more parameters of the first source model and/or the one or more parameters of the second source model and/or the one or more parameters of the third source model.

Example 17 provides the method according to Example 16, where the plurality of iterations further include modifying one or more parameters of the third source model based on the frequency-domain representation of the first signal.

Example 18 provides the method according to Example 11, where the heartbeat sensor includes an optical sensor configured to generate the first signal by detecting light at a first frequency or range of frequencies, and the method further includes performing processing applied to the first signal to a further signal generated by a further heartbeat sensor, the further heartbeat sensor including an optical sensor configured to generate the further signal by detecting light at a second frequency or range of frequencies, and the estimate of the heartbeat signal is computed further based on the further signal.

Example 19 provides the method according to Example 11, where the plurality of iterations are performed until a predefined maximum number of iterations is reached or until a divergence between the estimate of the time-frequency-domain representation for the first source (i.e. q(f,t|s1)) generated by the first source model and an updated estimate of the frequency-domain representation for the first source (i.e. p(f,t|s1)) satisfies a predefined criteria.

Example 20 provides the method according to Example 19, where the predefined criteria is based on a Kullback-Leibler (KL) divergence.

Example 21 provides the method according to Example 11, where performing the plurality of iterations includes carrying out an iterative algorithm based on a probabilistic inference approach.

Example 22 provides the method according to Example 11, further including processing the first signal and/or the second signal with a pre-processing filter to substantially attenuate signal content outside of a reasonable frequency band of interest corresponding to a range of reasonable heart rate frequencies.

Example 23 provides the method according to Example 22, where the pre-processing filter is a low-pass filter or a band-pass filter; and the reasonable frequency band of interest includes frequencies between 0.5 Hertz and 4 Hertz.

Example 24 provides the method according to Example 11, further including applying a filter or mask to or removing a portion of the first signal indicative of a saturation condition of the heartbeat sensor.

Example 25 provides the method according to Example 11, further including generating a time-frequency-domain representation of the estimate of the heartbeat signal computed based on the one or more parameters of the first source model and/or the one or more parameters of the second source model; and tracking one or more contours present in the time-frequency-domain representation to track the heartbeat signal.

Example 26 provides the method according to Example 11, where the heartbeat sensor includes one or more of the following: optical sensor, audio sensor, capacitive sensor, magnetic sensor, chemical sensor, humidity sensor, moisture sensor, pressure sensor, and biosensor.

Example 27 provides the method according to Example 11, where the frequency-domain representation of each signal includes a Short Time Fourier Transform (STFT).

Example 28 provides the method according to Example 11, where the time-frequency-domain representation of each signal includes a plurality of elements, each element including a value indicative of a magnitude of the signal associated with a different pair of frequency (f) and time (t) values or ranges.

Example 29 provides an apparatus for assisting separation of a heartbeat signal present in a first signal generated by a heartbeat sensor, the apparatus including at least one memory configured to store computer executable instructions, and at least one processor coupled to the at least one memory and configured, when executing the instructions, to perform the method according to any one of the preceding Examples.

Example 30 provides a non-transitory computer readable storage medium storing software code portions configured for, when executed on a processor, carrying out the method according to any one of the preceding Examples.

Example 31 provides an apparatus including means for performing the method according to any one of the preceding Examples.

Further Variations and Implementations

It is envisioned that a heart rate monitoring apparatus as described herein can be provided in many areas including medical equipment, security monitoring, patient monitoring, healthcare equipment, medical equipment, automotive equipment, aerospace equipment, consumer electronics, and sports equipment, etc.

In some cases, the heart rate monitoring apparatus can be used in professional medical equipment in a healthcare setting such as doctor's offices, emergency rooms, hospitals, etc. In some cases, the heart rate monitoring apparatus can be used in less formal settings, such as schools, gyms, homes, offices, outdoors, under water, etc. The heart rate monitoring apparatus can be provided in a consumer healthcare product.

The heart rate monitoring apparatus or parts thereof can take many different forms. Examples include watches, rings, wristbands, chest straps, headbands, headphones, ear buds, clamps, clips, clothing, bags, shoes, glasses, googles, hats, suits, necklace, attachments/patches/strips/pads which can adhere to a living being, accessories, portable devices, and so on. In particular, wearables technology (or referred often as “wearables”, i.e., electronics which are intended to be worn by humans or other living beings) can greatly leverage the benefits of the heart rate monitoring apparatus disclosed herein due to the wearables' portability and the heart rate monitoring technique's robustness against motion artifacts. Even in the presence of noise, the wearable can effectively track a heart rate. Besides wearables, portable or mobile devices such as mobile phones and tablets can also include a processor having the tracking functions, an analog front end, a light source and a light sensor (or an extension (wired or wireless) having the light source and light sensor) to provide a heart rate monitoring apparatus. Users can advantageously use a ubiquitous mobile phone to make a heart rate measurement. Furthermore, it is envisioned that the heart rate monitoring apparatus can be used in wired or wireless accessories such as cuffs, clips, straps, bands, probes, etc., to sense physiological parameters of a living being. These accessories can be connected to a machine configured to provide the processor and the analog front end. The analog front end could be provided in the accessory or in the machine.

Besides tracking a heart rate, the heart rate monitoring apparatus can be provided to sense or measure other physiological parameters such as oxygen saturation (SpO2), blood pressure, respiratory rate, activity or movement, etc. Besides humans, the heart rate monitoring apparatus can be provided to tracking frequencies present in signals sensing other living beings such as animals, insects, plants, fungi, etc.

In the discussions of the embodiments above, the capacitors, clocks, DFFs, dividers, inductors, resistors, amplifiers, switches, digital core, transistors, and/or other components can readily be replaced, substituted, or otherwise modified in order to accommodate particular circuitry needs. Moreover, it should be noted that the use of complementary electronic devices, hardware, software, etc. offer an equally viable option for implementing the teachings of the present disclosure. For instance, instead of processing the signals in the digital domain, it is possible to provide equivalent electronics that can process the signals in the analog domain.

In one example embodiment, any number of electrical circuits may be used to implement the iterative mask estimation techniques as described herein, and, in particular, to implement elements shown in the FIGUREs. Such electrical circuits may be implemented on a board of an associated electronic device. The board can be a general circuit board that can hold various components of the internal electronic system of the electronic device and, further, provide connectors for other peripherals. More specifically, the board can provide the electrical connections by which the other components of the system can communicate electrically. Any suitable processors (inclusive of digital signal processors, microprocessors, supporting chipsets, etc.), computer-readable non-transitory memory elements, etc. can be suitably coupled to the board based on particular configuration needs, processing demands, computer designs, etc. Other components such as external storage, additional sensors, controllers for audio/video display, and peripheral devices may be attached to the board as plug-in cards, via cables, or integrated into the board itself. In various embodiments, the functionalities described herein may be implemented in emulation form as software or firmware running within one or more configurable (e.g., programmable) elements arranged in a structure that supports these functions. The software or firmware providing the emulation may be provided on non-transitory computer-readable storage medium comprising instructions to allow a processor to carry out those functionalities. In some cases, application specific hardware can be provided with or in the processor to carry out those functionalities.

In another example embodiment, the electrical circuits of the FIGURES may be implemented as stand-alone modules (e.g., a device with associated components and circuitry configured to perform a specific application or function) or implemented as plug-in modules into application specific hardware of electronic devices. Note that particular embodiments of the present disclosure may be readily included in a system on chip (SOC) package, either in part, or in whole. An SOC represents an IC that integrates components of a computer or other electronic system into a single chip. It may contain digital, analog, mixed-signal, and often radio frequency functions: all of which may be provided on a single chip substrate. Other embodiments may include a multi-chip-module (MCM), with a plurality of separate ICs located within a single electronic package and configured to interact closely with each other through the electronic package. In various other embodiments, the varying an iterative mask estimation functionalities may be implemented in one or more silicon cores in Application Specific Integrated Circuits (ASICs), Field Programmable Gate Arrays (FPGAs), and other semiconductor chips.

Note that the activities discussed above with reference to the FIGURES are applicable to any integrated circuits that involve signal processing, particularly those that can execute specialized software programs, or algorithms, some of which may be associated with processing digitized real-time data to track a heart rate. Certain embodiments can relate to multi-DSP signal processing, floating point processing, signal/control processing, fixed-function processing, microcontroller applications, etc. In certain contexts, the features discussed herein can be applicable to medical systems, scientific instrumentation, wireless and wired communications, radar, industrial process control, audio and video equipment, current sensing, instrumentation (which can be highly precise), and other digital-processing-based systems. Moreover, certain embodiments discussed above can be provisioned in digital signal processing technologies for medical imaging, patient monitoring, medical instrumentation, and home healthcare. This could include pulmonary monitors, heart rate monitors, pacemakers, etc. Other applications can involve automotive technologies for safety systems (e.g., stability control systems, driver assistance systems, braking systems, infotainment and interior applications of any kind). Furthermore, powertrain systems (for example, in hybrid and electric vehicles) can use high-precision data conversion products in battery monitoring, control systems, reporting controls, maintenance activities, etc. It is envisioned that these applications can also utilize the disclosed iterative mask estimation techniques. In yet other example scenarios, the teachings of the present disclosure can be applicable in the industrial markets that include process control systems aiming to track a varying frequency to help drive productivity, energy efficiency, and reliability.

Note that with the numerous examples provided herein, interaction may be described in terms of two, three, four, or more parts. However, this has been done for purposes of clarity and example only. It should be appreciated that the system can be consolidated in any suitable manner. Along similar design alternatives, any of the illustrated components, modules, and elements of the FIGURES may be combined in various possible configurations, all of which are clearly within the broad scope of this Specification. In certain cases, it may be easier to describe one or more of the functionalities of a given set of flows by only referencing a limited number of electrical elements. It should be appreciated that the features of the FIGURES and its teachings are readily scalable and can accommodate a large number of components, as well as more complicated/sophisticated arrangements and configurations. Accordingly, the examples provided should not limit the scope or inhibit the broad teachings of the electrical circuits as potentially applied to a myriad of other architectures.

Note that in this Specification, references to various features (e.g., elements, structures, modules, components, steps, operations, parts, characteristics, etc.) included in “one embodiment”, “example embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments.

It is also important to note that the functions related to iterative mask estimation techniques described herein, illustrate only some of the possible tracking functions that may be executed by, or within, systems illustrated in the FIGURES. Some of these operations may be deleted or removed where appropriate, or these operations may be modified or changed considerably without departing from the scope of the present disclosure. In addition, the timing of these operations may be altered considerably. The preceding operational flows have been offered for purposes of example and discussion. Substantial flexibility is provided by embodiments described herein in that any suitable arrangements, chronologies, configurations, and timing mechanisms may be provided without departing from the teachings of the present disclosure. Note that all optional features of the apparatus described above may also be implemented with respect to the method or process described herein and specifics in the examples may be used anywhere in one or more embodiments.

The ‘means for’ in these instances (above) can include (but is not limited to) using any suitable component discussed herein, along with any suitable software, circuitry, hub, computer code, logic, algorithms, hardware, controller, interface, link, bus, communication pathway, etc. In a second example, the system includes memory that further comprises machine-readable instructions that when executed cause the system to perform any of the activities discussed above.

Numerous other changes, substitutions, variations, alterations, and modifications may be ascertained to one skilled in the art and it is intended that the present disclosure encompass all such changes, substitutions, variations, alterations, and modifications as falling within the scope of the appended claims.

Although the claims are presented in single dependency format in the style used before the USPTO, it should be understood that any claim can depend on and be combined with any preceding claim of the same type unless that is clearly technically infeasible.

Note that all optional features of the apparatus described above may also be implemented with respect to the method or process described herein and specifics in the examples may be used anywhere in one or more embodiments.

What is claimed is:

1. A computer-implemented method for assisting separation of a heartbeat signal present in a first signal generated by a heartbeat sensor, the method comprising:
processing the first signal to compute a time-frequency-domain representation (p_{obs}(f,t)) of the first signal;
processing a second signal to compute a time-frequency-domain representation (p_{accx}(f,t)) of the second signal, the second signal indicative of a motion of the heartbeat sensor with respect to a first direction (x);
initializing a first source model for a first source (s₁), the first source representing a source of the heartbeat signal;
initializing a second source model for a second source (s₂), the second source representing a source of a contribution to the first signal due to the motion of the heartbeat sensor with respect to the first direction, wherein the second source model is initialized based on the time-frequency-domain representation (p_{accx}(f,t)) of the second signal;
performing a plurality of iterations of modifying one or more parameters of the first source model and/or one or more parameters of the second source model based on the time-frequency-domain representation of the first signal; and
following the plurality of iterations, computing an estimate of the heartbeat signal based on the one or more parameters of the first source model and/or the one or more parameters of the second source model.

2. The method according to claim 1, wherein each iteration of the plurality of iterations comprises:
using the first source model to generate an estimate of a time-frequency-domain representation (q(f,t|s₁)) for the first source,
using the second source model to generate an estimate of a time-frequency-domain representation (q(f,t|s₂)) for the second source, and
based on the estimate of the time-frequency-domain representation for the first source and the estimate of the time-frequency-domain representation for the second source, computing a first mask function q(s₁|f,t) for separating the heartbeat signal from the first signal and computing a second mask function q(s₂|f,t) for separating, from the first signal, the contribution due to motion of the heartbeat sensor with respect to the first direction.

3. The method according to claim 2, wherein said each iteration of the plurality of iterations further comprises:
applying the first mask function to the time-frequency-domain representation of the first signal to generate an updated estimate of a time-frequency-domain representation (p(f,t|s₁)) for the first source,
applying the second mask function to the time-frequency-domain representation of the first signal to generate an updated estimate of a frequency-domain representation for the second source (s2),
applying the updated estimate of the time-frequency-domain representation for the first source to update one or more parameters of the first source model, and
applying the updated estimate of the frequency-domain representation for the second source to update one or more parameters of the second source model.

4. The method according to claim 3, wherein:
applying the first mask function comprises performing a point-wise multiplication of the first mask function and the time-frequency-domain representation of the first signal, and
applying the second mask function comprises performing a point-wise multiplication of the second mask function and the time-frequency-domain representation of the first signal.

5. The method according to claim 1, wherein:
the first source model comprises a Non-negative Tensor Factorization (NTF) source model or a one-peak source model, and/or
the second source model comprises the NTF source model or a constant source model.

6. The method according to claim 1, further comprising:
processing a third signal to compute a time-frequency-domain representation (p_{accy}(f,t)) of the third signal, the third signal indicative of a motion of the heartbeat sensor with respect to a second direction (y); and
initializing a third source model for a third source (s3), the third source representing a source of a contribution to the first signal due to the motion of the heartbeat sensor with respect to the second direction, wherein the third source model is initialized based on the time-frequency-domain representation (p_{accy}(f,t)) of the third signal,
wherein the estimate of the heartbeat signal is computed based on the one or more parameters of the first source model and/or the one or more parameters of the second source model and/or the one or more parameters of the third source model.

7. The method according to claim 6, wherein the plurality of iterations further include modifying one or more parameters of the third source model based on the frequency-domain representation of the first signal.

8. The method according to claim 1, wherein:
the heartbeat sensor comprises an optical sensor configured to generate the first signal by detecting light at a first frequency or range of frequencies, and
the method further comprises performing processing applied to the first signal to a further signal generated by a further heartbeat sensor, the further heartbeat sensor comprising an optical sensor configured to generate the further signal by detecting light at a second frequency or range of frequencies, and
the estimate of the heartbeat signal is computed further based on the further signal.

9. The method according to claim 1, wherein the plurality of iterations are performed until a predefined maximum number of iterations is reached or until a divergence between the estimate of the time-frequency-domain representation for the first source (i.e. q(f,t|s1)) generated by the first source model and an updated estimate of the frequency-domain representation for the first source (i.e. p(f,t|s1)) satisfies a predefined criteria.

10. The method according to claim 9, wherein the predefined criteria is based on a Kullback-Leibler (KL) divergence.

11. The method according to claim 1, wherein performing the plurality of iterations comprises carrying out an iterative algorithm based on a probabilistic inference approach.

12. The method according to claim 1, further comprising:
processing the first signal and/or the second signal with a pre-processing filter to substantially attenuate signal content outside of a reasonable frequency band of interest corresponding to a range of reasonable heart rate frequencies.

13. The method according to claim 12, wherein:
the pre-processing filter is a low-pass filter or a band-pass filter; and
the reasonable frequency band of interest comprises frequencies between 0.5 Hertz and 4 Hertz.

14. The method according to claim 1, further comprising:
applying a filter or mask to or removing a portion of the first signal indicative of a saturation condition of the heartbeat sensor.

15. The method according to claim 1, further comprising:
generating a time-frequency-domain representation of the estimate of the heartbeat signal computed based on the one or more parameters of the first source model and/or the one or more parameters of the second source model; and
tracking one or more contours present in the time-frequency-domain representation to track the heartbeat signal.

16. The method according to claim 1, wherein the heartbeat sensor comprises one or more of the following: optical sensor, audio sensor, capacitive sensor, magnetic sensor, chemical sensor, humidity sensor, moisture sensor, pressure sensor, and biosensor.

17. The method according to claim 1, wherein the frequency-domain representation of each signal comprises a Short Time Fourier Transform (STFT).

18. The method according to claim 1, wherein the time-frequency-domain representation of each signal comprises a plurality of elements, each element comprising a value indicative of a magnitude of the signal associated with a different pair of frequency (f) and time (t) values or ranges.

19. An apparatus for assisting separation of a heartbeat signal present in a first signal generated by a heartbeat sensor, the apparatus comprising:
at least one memory configured to store computer executable instructions, and
at least one processor coupled to the at least one memory and configured, when executing the instructions, to:
process the first signal to compute a time-frequency-domain representation (p_{obs}(f,t)) of the first signal;
process a second signal to compute a time-frequency-domain representation (p_{accx}(f,t)) of the second signal, the second signal indicative of a motion of the heartbeat sensor with respect to a first direction (x);
initialize a first source model for a first source (s₁), the first source representing a source of the heartbeat signal;
initialize a second source model for a second source (s₂), the second source representing a source of a contribution to the first signal due to the motion of the heartbeat sensor with respect to the first direction, wherein the second source model is initialized based on the time-frequency-domain representation (p_{accx}(f,t)) of the second signal;
perform a plurality of iterations of modifying one or more parameters of the first source model and/or one or more parameters of the second source model based on the time-frequency-domain representation of the first signal; and
following the plurality of iterations, compute an estimate of the heartbeat signal based on the one or more parameters of the first source model and/or the one or more parameters of the second source model.

20. A non-transitory computer readable storage medium storing software code portions configured, when executed on a processor, to:
process the first signal to compute a time-frequency-domain representation (p_{obs}(f,t)) of the first signal;
process a second signal to compute a time-frequency-domain representation (p_{accx}(f,t)) of the second signal, the second signal indicative of a motion of the heartbeat sensor with respect to a first direction (x);
initialize a first source model for a first source (s₁), the first source representing a source of the heartbeat signal;
initialize a second source model for a second source (s₂), the second source representing a source of a contribution to the first signal due to the motion of the heartbeat sensor with respect to the first direction, wherein the second source model is initialized based on the time-frequency-domain representation (p_{accx}(f,t)) of the second signal;
perform a plurality of iterations of modifying one or more parameters of the first source model and/or one or more parameters of the second source model based on the time-frequency-domain representation of the first signal; and
following the plurality of iterations, compute an estimate of the heartbeat signal based on the one or more parameters of the first source model and/or the one or more parameters of the second source model.
